(19) United States
(12) Patent Application Publication
(10) Pub. No.: US 2024/0148038 A1
(43) Pub. Date: May 9, 2024

(54) TASTE-MASKING COMPOUNDS AND COMPOSITIONS AND USES THEREOF

(71) Applicant: HEALTHTECH BIO ACTIVES, S.L.U., BARCELONA (ES)

(72) Inventors: Francisco Javier CRESPO MONTERO, BARCELONA (ES); Tom Nelly Aime D'HOORE, BARCELONA (ES)

(73) Assignee: HEALTHTECH BIO ACTIVES, S.L.U., BARCELONA (ES)

(21) Appl. No.: 18/277,751

(22) PCT Filed: Feb. 17, 2022
(86) PCT No.: PCT/EP2022/053946

§ 371 Date: Aug. 17, 2023

(30) Foreign Application Priority Data
Feb. 18, 2021 (EP) 21382129.1

(51) Int. Cl. A23L 27/00; A21D 2/14; A23C 9/152; A23G 1/32; A23L 2/02; A23L 2/56; C07H 17/04

(52) U.S. Cl. CPC A23L 27/84; A21D 2/14; A23C 9/152; A23G 1/32; A23L 2/02; A23L 2/56; A23L 27/86; C07H 17/04

(57) ABSTRACT

tract: The present invention relates to the use of a compound of Formula (I) or a stereoisomer or salt thereof, wherein R1 is a saccharide consisting of 1 or 2 monosaccharide units as taste masker and to taste-masking compositions containing said compounds.

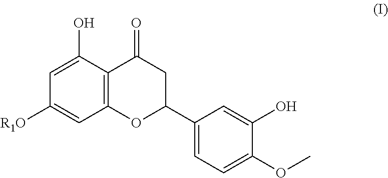

This application claims the benefit of European Patent Application EP21382129.1 filed 18 Feb. 2021.

### TECHNICAL FIELD

The present invention relates to the field of taste-masking, in particular, it relates to taste-making compounds and to taste-masking compositions containing said compounds.

### BACKGROUND ART

There are a number of substances which are frequently found in food, beverages and pharmaceutical products producing an unpleasant taste. Although many of said substances are naturally occurring in said products, like citrus fruits, coffee or tea, they can greatly decrease their value due to said unpleasant taste. Indeed, only rarely consumers accept the bitter and/or astringent taste of food and beverages, for example, in black coffee, black or green tea, beer, red wine, grapefruit products or bitter lemon. In most other cases, those tastes are not desirable and must be eliminated or masked and thus, subsequent treatment is necessary.

Many substances regarded as healthy and purposely added to healthy food preparations are however perceived adversely in terms of taste by the consumers. This may be the case of the bitter taste of certain vitamins, minerals, peptides or protein hydrolysates or the bitter or astringent taste of certain plant-based phenols, flavonoids, isoflavones, terpenes and glucosinolates, which are reported to have positive antioxidant and anticarcinogenic properties.

Another example is the bitter aftertaste associated to potassium chloride, which is increasingly being used to provide saltiness to food products, as a healthier sodium chloride replacer.

A particularly problematic group of consumables in terms of taste are pharmaceutical products, which frequently contain active ingredients having bitter, astringent or metallic tastes. This adverse perception may even adversely affect the patient compliance, especially in those population groups which are particularly sensitive to bad tastes, particularly, children.

Consequently, since the taste of the consumable product plays an important role in their acceptance by the consumer, efforts have been made to suppress or reduce those unpleasant tastes in consumables, namely, in food, beverages and pharmaceutical products.

It is, therefore, desirable to find substances, preferably natural or nature-identical substances, which can effectively suppress, or at least decrease, the bitter, sour, pungent, or unpleasant taste.

Depending on each particular substance and on each particular consumable product, different taste-masking solutions may be used, as have been extensively reported in the prior art, for example, some possible strategies are partly removing the bittering substances, coating or microencapsulating them, adding flavours and/or sweeteners, or adding specific taste-masking substances.

The use of taste-masking substances, which are able to modulate, reduce or suppress bitterness, sourness, pungent, astringency or metallic tastes, has proved to be useful for many particular applications.

A good number of substances have been reported in the art for use as taste-masking agents in food and pharmaceuticals. Examples are some sweeteners, such as thaumatin and neohesperidin dihydrochalcone; polymers and complexing agents, such as cyclodextrins, poly-γ-glutamic acid and chitosan; neodiosmin; L-ornithine and derivatives such as L-ornithyl-β-alanine or L-ornithinyltaurine; several dipeptides containing aspartic acid such L-aspartyl-L-phenylalanine potassium salt; sodium salts of saturated fatty acids, such as sodium stearate, palmitate and laurate; organic phosphates, phosphonates, vanadates, thiophosphates, and biphosphates; flavanones such as eriodictyol and homoeriodictyol; among many others.

Sodium chloride exhibits a bitter-masking effect against many bitter substances (Breslin & Beauchamp, 1997, Nature; vol. 387, p. 563); however, the intake of relatively large amounts of salt can lead, for example, to cardiovascular disorders.

Neodiosmin and diosmin have been reported as bitter masking substances for instance by Raithore Smita et al., 2020; in Journal of Agricultural and Food Chemistry; vol. 68; pp. 1038-1050. The reduction of bitterness by addition of neodiosmin has also been reported in WO91/18523A1 which discloses the neodiosmin as suitable bitterness inhibitor or in U.S. Pat. No. 4,031,265A which discloses the reduction of bitterness in citrus juices by neodiosmin.

Also, neohesperidin dihydrochalcone has been reported as an effective taste-masking substance, for example, it has been used for masking the bitter taste of substances like paracetamol, dextromethorphan and other pharmaceuticals, as well as in special foods, as disclosed in Borrego et al., Neohesperidin Dihydrochalcone, in: L O'Brien Nabors, eds. Alternative Sweeteners, Fourth Edition, CRC Press, 2012, 94-95.

However, achieving an acceptable level of taste-masking usually requires the use of considerable amounts of the taste-masking substance, which is unsatisfactory in terms of costs and additional off-notes.

Therefore, there is a need in the art for new taste-masking compounds and compositions, preferably natural, which are able to effectively modulate, reduce or suppress unpleasant tastes and require the use of reduced amounts of the taste-masking compound or composition.

### BRIEF DESCRIPTION OF DRAWINGS

FIG. 1 is a spider diagram representing the organoleptic profile of orange juices containing oleuropeins wherein hesperidin 2S at two concentrations: 100 ppm (FIG. 1A) and 200 ppm (FIG. 1B) has been added. The sensory descriptors evaluated were acidity, bitterness, astringency, orange notes, flavonoid notes, woody notes, mouthfeel, off-flavour: woody, flavonoid, persistence and dryness, on a 0-5 point scale (0=none, 1=very weak, 2=weak, 3=moderate, 4=strong, 5=very strong).

FIG. 2 is a spider diagram representing the organoleptic profile of potassium-chloride containing crackers wherein hesperidin 2S at 200 ppm has been added (FIG. 2). The sensory descriptors evaluated were saltiness, metallic, pungent, and lingering, on a 0-5 point scale (0=none, 1=very weak, 2=weak, 3=moderate, 4=strong, 5=very strong).

FIG. 3 is a spider diagram representing the organoleptic profile of dark chocolate (95%) containing 200 ppm hesperidin 2S (FIG. 3). The sensory descriptors evaluated were cocoa notes, bitterness, astringency, flavonoid notes, mouthfeel, lingering, off-flavour: flavonoid, on a 0-5 point scale (0=none, 1=very weak, 2=weak, 3=moderate, 4=strong, 5=very strong).

FIG. 4 is a spider diagram representing the organoleptic profile of orange juices from oranges affected by greening disease and containing hesperidin 2S at two concentrations: 100 ppm (FIG. 4A) and 200 ppm (FIG. 4B). The sensory descriptors evaluated were initial sweetness, acidity, bitterness, astringency, orange notes, flavonoid notes, body, bitter aftertaste, green orange notes, and cooked note, on a 0-5 point scale (0=none, 1=very weak, 2=weak, 3=moderate, 4=strong, 5=very strong).

FIG. 5 is a spider diagram representing the organoleptic profile of fresh squeezed orange juices wherein hesperidin 2S at two concentrations: 100 ppm (FIG. 5A) and 200 ppm (FIG. 5B) has been added. The sensory descriptors evaluated were sweetness, acidity, bitterness, astringency, orange notes, mouthfeel and lingering, on a 0-5 point scale (0=none, 1=very weak, 2=weak, 3=moderate, 4=strong, 5=very strong).

FIG. 6 is a spider diagram representing the organoleptic profile of tonic water containing hesperidin 2S at two concentrations: 100 ppm (FIG. 6A) and 200 ppm (FIG. 6B) has been added. The sensory descriptors evaluated were global sweetness, acidity, lemon-lime notes, astringency, bitterness and mouthfeel on a 0-5 point scale (0=none, 1=very weak, 2=weak, 3=moderate, 4=strong, 5=very strong).

FIG. 7 is a spider diagram representing the organoleptic profile of a plant-based burger containing oleuropeins wherein hesperidin 2S at two concentrations: 100 ppm (FIG. 7A) and 200 ppm (FIG. 7B) has been added. The sensory descriptors evaluated were juiciness, firmness, elasticity, fat texture, meaty notes, bean notes, bitterness, flavonoid notes and woody-walnut notes, on a 0-5 point scale (0=none, 1=very weak, 2=weak, 3=moderate, 4=strong, 5=very strong).

FIG. 8 is a spider diagram representing the organoleptic profile of protein milkshake wherein hesperidin 2S at two concentrations: 100 ppm (FIG. 5A) and 200 ppm (FIG. 5B) has been added. The sensory descriptors evaluated were global sweetness, cocoa notes, dry fava notes, powdery, astringency, bitterness and mouthfeel on a 0-5 point scale (0=none, 1=very weak, 2=weak, 3=moderate, 4=strong, 5=very strong).

### SUMMARY OF THE INVENTION

It is an object of the present invention to find readily accessible natural substances which exhibit a taste-masking effect, in particular, substances which can be used for masking or reducing the bitter, sour, pungent, or unpleasant taste impression of a substance causing bitter, sour, pungent, or unpleasant taste impression in an ingestible product.

Therefore, in a first aspect, the invention relates to the use of a compound of the general formula (I) or a stereoisomer or a salt thereof

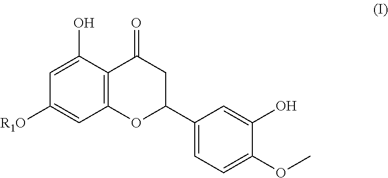

wherein R1 is a saccharide consisting of 1 or 2 monosaccharide units;
   for masking or reducing the bitter, sour, pungent, or unpleasant taste impression of a substance causing bitter, sour, pungent, or unpleasant taste impression in an ingestible product;
   wherein said compound of formula (I) is not used in combination or as admixture with a hesperidin glycoside.

A second aspect of the invention relates to a taste masking composition comprising a compound of formula (I)

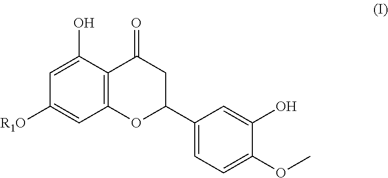

wherein R1 is saccharide consisting of 2 monosaccharide units and the (2S)- and (2R)-enantiomers are present in a weight ratio of 99:1 to 80:20, preferably of 99:1 to 90:10; and wherein said composition does not contain a hesperidin glycoside.

### DETAILED DESCRIPTION OF THE INVENTION

The authors of the present invention have found that a compound of formula (I) according to the present invention can surprisingly be used as a taste-masking compound, particularly, it can be used for masking or reducing the bitter, sour, pungent, or unpleasant taste impression of a substance causing bitter, sour, pungent, or unpleasant taste impression in an ingestible product.

Thus, in a first aspect, the invention relates to the use of a compound of the general formula (I) or a stereoisomer or a salt thereof

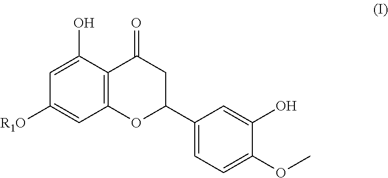

wherein R1 is a saccharide consisting of 1 or 2 monosaccharide units;
   for masking or reducing the bitter, sour, pungent, or unpleasant taste impression of a substance causing bitter, sour, pungent, or unpleasant taste impression in an ingestible product;
   wherein said compound of formula (I) is not used in combination or as admixture with a hesperidin glycoside.

Along the present description, as well as in the claims, the singular forms, usually preceded by “a”, “an” or “the”, are to be construed to include also the plural, unless the context clearly indicates otherwise.

As used herein, the term “about” used before a quantitative value is to be construed as including the specific quantitative value and also a variation of no more than ±10% of the given value, preferably of no more than ±5% of the given value.

As used herein, the term “taste-masking agent”, “taste masking compound”, or “taste masker”, refers to any substance, compound or composition providing a taste-masking effect. A taste-masking effect, as is well known in the art, means the perceived reduction, modulation, or elimination of an unpleasant taste or unpleasant taste impression of a substance. In particular, the compound of the invention is a substance which is able to mask or reduce the bitter, sour, pungent, or unpleasant taste impression of a substance causing bitter, sour, pungent, or unpleasant taste impression in an ingestible product.

Typically, for exerting the taste-masking effect, the taste-masking compound or composition is mixed with the unpleasantly tasting substance, generally, both contained in an ingestible or edible product. Alternatively, the compound of formula (I) according to the invention or the taste-masking composition according to the invention, can be prepared in situ, so both components, i.e., the compound of formula (I) according to the invention or taste masking composition according to the invention may be added to an unpleasantly tasting substance, in particular, to a substance causing bitter, sour, pungent, or unpleasant taste impression or to an ingestible product comprising an unpleasantly tasting substance, typically followed by thoroughly mixing.

In a particular embodiment, the compound of Formula (I) to be used according to the invention is a compound of formula (Ia):

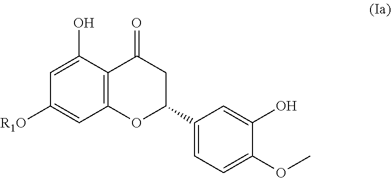

wherein R1 is a saccharide consisting of 2 monosaccharide units.

In a preferred embodiment, the compound of formula (I) or (Ia) is (2S)-5-hydroxy-2-(3-hydroxy-4-methoxyphenyl)-7-[(2S,3R,4S,5S,6R)-3,4,5-trihydroxy-6-{[(2R,3R,4R,5R,6S)-3,4,5-trihydroxy-6-methyloxan-2-yl]oxymethyl}oxan-2-yl]oxy-2,3-dihydrochromen-4-one.

In another particular embodiment, the compound of formula (I) according to the invention is used as a mixture of the (2S)- and (2R)-enantiomers, in any mixing ratio. Preferably, when used as a mixture of the two enantiomers, the (2S)- and (2R)-enantiomers are present in a weight ratio of 99:1 to 70:30, more preferably of 99:1 to 80:20, even more preferably of 99:1 to 90:10. In a preferred embodiment, the weight ratio is of 99:1 to 93:7.

In another particular embodiment, the compound of formula (I) according to the invention is used as a taste masking composition, wherein said taste masking composition does not contain a hesperidin glycoside. In a more particular embodiment, the (2S)- and (2R)-enantiomers of the compound of formula (I) are contained in said taste masking composition in a weight ratio of weight ratio of 99:1 to 70:30, preferably of 99:1 to 80:20, more preferably of 99:1 to 90:10. In an even more preferred embodiment, the weight ratio is of 99:1 to 93:7.

Thus, in another aspect, the invention refers to a taste masking composition comprising a compound of formula (I)

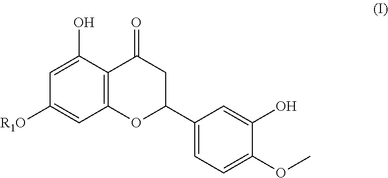

wherein R1 is saccharide consisting of 2 monosaccharide units and the (2S)- and (2R)-enantiomers are present in a weight ratio of 99:1 to 80:20, preferably of 99:1 to 90:10; and wherein said composition does not contain a hesperidin glycoside.

In a particular embodiment, said compound of formula (I) to be used in said taste masking composition of the invention is hesperidin. In a more preferred embodiment, said compound is (2S)-5-hydroxy-2-(3-hydroxy-4-methoxyphenyl)-7-[(2S,3R,4S,5S,6R)-3,4,5-trihydroxy-6-{[(2R,3R,4R,5R,6S)-3,4,5-trihydroxy-6-methyloxan-2-yl]oxymethyl}oxan-2-yl]oxy-2,3-dihydrochromen-4-one.

Said taste masking composition according to the invention might comprise other taste masking compounds, not including hesperidin glycosides. In a particular embodiment, other taste masking compounds which can be used in said composition are, for example, naringenin, eriodictyol, eriodictyol 7-methyl ether, eriodictyol 5-methyl ether, homoeriodictyol, phloretin, rutin, quercitin, isoquercitrin, and/or salts, derivatives and/or mixtures thereof. In a particular embodiment, the composition contains a compound of formula (I) according to the invention in combination with naringenin. In another particular embodiment, the composition contains a compound of formula (I) according to the invention in combination with phloretin. In another particular embodiment, the composition contains a compound of formula (I) according to the invention in combination with eriodictyol. In another particular embodiment, the composition contains a compound of formula (I) according to the invention in combination with homoeriodictyol. In another particular embodiment, the composition contains a compound of formula (I) according to the invention in combination with rutin. In another particular embodiment, the composition contains a compound of formula (I) according to the invention in combination with quercitin. In another particular embodiment, the composition contains a compound of formula (I) according to the invention in combination with isoquercitrin.

In a further aspect, the present invention relates to a method of masking or reducing the bitter, sour, pungent, or unpleasant taste impression of a substance causing bitter, sour, pungent, or unpleasant taste impression in an ingestible product, said method comprising the step of adding a compound of formula (I) according to the invention or a taste masking composition comprising the compound of formula (I) according to the invention, to the substance causing said bitter, sour, pungent, or unpleasant taste impression. Typically, the compound of formula (I) according to the invention or composition comprising the compound of formula (I) according to the invention is added to said substance in a taste-masking effective amount.

As shown in the Examples accompanying the present invention, it has been found that compounds of formula (I) show a strong taste-masking activity. Indeed, the compounds of formula (I) according to the invention can, surprisingly, reduce and/or mask the bitter, sour, pungent or unpleasant taste impression of substances causing bitter, sour, pungent or unpleasant taste in a variety of ingestible products.

The bitter masking effect of a racemic hesperidin and a non-racemic hesperidin (H2S) against paracetamol was assayed. As it has been shown in Example 1, both compounds were able to reduce the unpleasant taste impressions of paracetamol solutions. Especially good results were obtained with H2S (93% hesperidin 2S: 7% hesperidin 2R) which was able to reduce paracetamol bitterness to a greater extent than the already good bitterness reduction obtained with racemic hesperidin. Moreover, comparative test for bitterness reduction using a different flavonoid compound, neodiosmin, was also assayed. The results showed that both a racemic hesperidin and a non-racemic hesperidin (H2S) reduced bitterness to a greater extent than neodiosmin, being the reduction much greater in the case of non-racemic hesperidin (See Table 2-1 and Table 2-3).

In Example 2, it is shown that the woody and bitter taste coming from the addition of olive polyphenols, in particular, oleuropein, to an orange juice are masked when a compound of formula (I) according to the invention is added to the orange juice containing oleuropein. In particular, the compound (2S)-5-hydroxy-2-(3-hydroxy-4-methoxyphenyl)-7-[(2S,3R,4S,5S,6R)-3,4,5-trihydroxy-6-{[(2R,3R,4R,5R,6S)-3,4,5-trihydroxy-6-methyloxan-2-yl]oxymethyl}oxan-2-yl]oxy-2,3-dihydrochromen-4-one (2S-hesperidin or H2S) is added as a H₂O—NaOH solution to an orange juice sample containing oleuropein. The results show a clear reduction of the bitter and woody taste. Further, it is observed that the orange notes are enhanced.

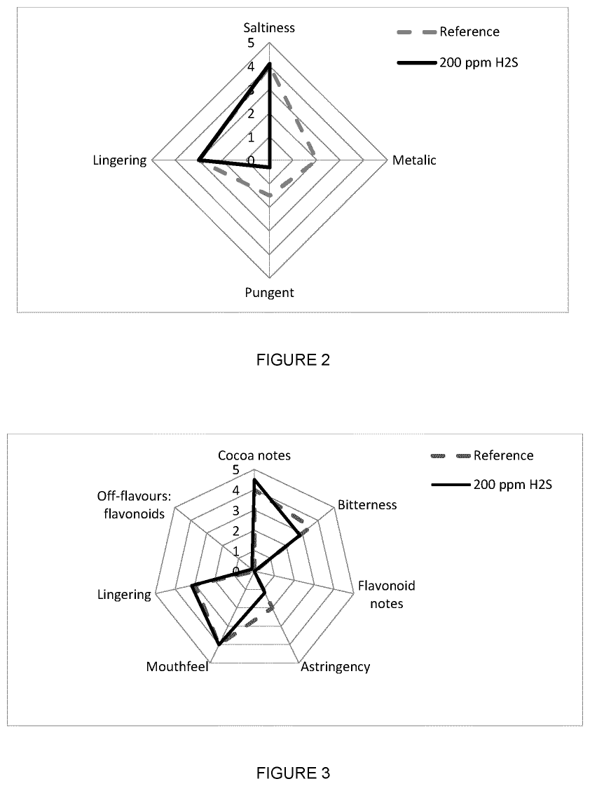

In Example 3, it is shown that the metallic and pungent notes coming from the addition of KCl to crackers, are suppressed when a compound of formula (I) according to the invention is added to the crackers. Moreover, as it is shown in FIG. 2, the addition of the compound H2S to the crackers, results in an increase of saltiness and overall, giving a more rounded taste of the cracker.

The inventors have also shown that the addition of a compound of formula (I), according to the invention, in particular H2S, to a dark chocolate sample (Example 4) surprisingly, decreases bitterness and astringency. Moreover, as it can be seen in FIG. 3, the cocoa notes are slightly enhanced and flavonoid notes are not present.

The inventors have also tested the effect of the compound of formula (I) according to the present invention on a limonin solution (Example 5). Citrus greening disease (or HLB) is a disease of citrus caused by a vector-transmitted pathogen. Affected trees produce small, irregularly shaped fruits with a thick, pale peel that tastes very bitter. One the main causes of the characteristic bitter taste, is the increase of two molecules: limonin and nomilin. The results clearly show that addition of a compound according to the invention, in particular, H2S, to a limonin solution produces a significant debittering effect (see Table 7).

Moreover, the effect of hesperidin on orange juices obtained from greening afflicted oranges has also been tested. As it is shown in Example 6 below, addition of hesperidin to orange juices obtained from greening afflicted oranges, surprisingly produce a significant reduction of bitterness, acidity and astringency.

Thus, in a particular embodiment of the invention, said substance causing bitter, sour, pungent or unpleasant taste is a substance which is naturally occurring in said ingestible product. In a more particular embodiment, said substance is a substance present in a citrus fruit of a plant suffering from Greening disease. More particularly, said citrus fruit is an orange. In a more particular embodiment, said substance is a polymethoxylated flavone, such as nobiletin and tangeretin, limonin, nomilin, hesperidin, or mixtures thereof.

In a particular embodiment of the invention, said ingestible product is a food product. Preferably, said food product is a beverage product. In a preferred embodiment, said beverage is a citrus juice. In a particular embodiment of the invention, said citrus juice is a juice produced from a citrus fruit plant suffering from Greening disease.

In another particular embodiment, the compound of formula (I) according to the invention is used for masking or reducing the bitter taste impression of a substance causing bitter taste impression in a citrus juice, particularly, in an orange juice obtained from fruits of plants suffering from Greening disease.

The bitter taste or bitterness is believed to be produced by certain substances which bind to special bitter taste receptors, located on the apical membrane of the taste receptor cells located in the taste buds on the tongue. The bitterness receptors are believed to be members of the G protein-coupled receptor (GPCR) superfamily and are referred to as T2Rs. These bitter receptors can interact with ligands which are chemically very diverse, so there is a large number of substances which are described as bitter, whose chemical structures may belong to many different chemical classes.

A non-limiting list of bitter substances includes, for example, flavanones, such as naringin, neohesperidin or hesperidin; flavones such as tangeritin or nobiletin; flavonols, such as avicularin, quercetin, quercitrin, isoquercetin, myricetin, or rutin; flavanols, such as taxifolin, catechins (catechin, epicatechin, epicatechin gallate, epigallocatechin, or epigallocatechin gallate) or theaflavins (theaflavin, isotheaflavin, neotheaflavin, theaflavin-3-gallate, theaflavin-3′-gallate, theaflavin-3,3′-digallate, isotheaflavin-3-gallate or theaflavic acid); isoflavones, such as genistein or daidzein; chalcones, such as phloridzin, triterpenes, such as limonin, nomilin or limonin glucoside; hydroxycinnamic acids such as caffeic acid or esters thereof; olive polyphenols such as oleuropein, glucosinolates, such as sinigrin, progoitrin or glucobrassicin; alkaloids, such as nicotine, theobromine, theophylline, quinine or caffeine; phenolic glycosides, such as salicin or arbutin; bitter alpha-acids found in the resin of hops, such as humulone, adhumulone, cohumulone, posthumulone, or prehumulone; metal salts, in particular potassium, magnesium and bismuth salts, such as potassium chloride, potassium gluconate, potassium carbonate, potassium succinate, potassium lactate, potassium malate; bismuth subcitrate, bismuth citrate, bismuth subgalate, bismuth salicylate or magnesium sulfate; some pharmaceutical active ingredients, such as fluoro-quinolone antibiotics, aspirin, ibuprofen, paracetamol, dextromethorphan, phenylephrine, loperamide, tramadol, ranitidine hydrochloride, acetylcysteine, glucosamine sulfate, erythromycin, levosulpiride, chlorhexidine, diosmin, β-lactam antibiotics, ambroxol, or guaifenesin; unsaturated fatty acids; vitamins; bitter tasting amino acids (e.g. leucine, isoleucine, valine, tryptophan, proline, histidine, tyrosine, lysine or phenylalanine) and some bitter-tasting peptides, particularly those containing the hydrophobic amino acids phenylalanine, tyrosine, tryptophan, leucine, isoleucine and valine.

In general, the taste of any substance known to impart a bitter taste may be masked using the compound of formula (I) according to the present invention or the composition of the invention. In a particular embodiment, when used specifically for masking bitterness, the taste masking compound or composition according to the invention may be referred to as anti-bittering agent, or bitter-masking agent.

In a particular embodiment of the invention, said substance causing bitter, sour, pungent or unpleasant taste is selected from the group consisting of caffeine, theobromine, theophylline, methylxanthine, quinine, brucine, strychnine, nicotine, salicin, arbutin, neohesperidin, hesperidin, naringin, quercitrin, rutin, phloridzin, pentagalloylglucose, galloylated catechols or epicatechols, proanthyocyanidines or procyanidines, thearubigin, quercetin, taxifolin, myricetin, salicin, gamma-oryzanol, caffeic acid or esters thereof, limonin, nomilin, a polymethoxylated flavone, absinthin, amarogentin, magnesium salts, calcium salts, potassium chloride, potassium gluconate, potassium carbonate, potassium sulfate, potassium lactate, potassium glutamate, potassium succinate, potassium malate, sodium sulfate, magnesium sulfate, fluoroquinolone antibiotics, paracetamol, ibuprofen, aspirin, beta-lactam antibiotics, ambroxol, propylthiouracil, guaifenesin, vitamin H, vitamin B1, vitamin B2, vitamin B6, vitamin B12, niacin, pantothenic acid, denatonium benzoate, sucralose octaacetate, leucine, isoleucine, valine, tryptophan, proline, histidine, tyrosine, lysine, phenylalanine, soy protein, whey protein, pea protein, rice protein, hemp protein, chia protein, wheat protein, potato protein, corn protein, sunflower protein, aspartame, steviol glycosides, monkfruit, collagen peptides, cannabinoids, and mixtures thereof.

In another particular embodiment, said substance causing said bitter, sour, pungent, or unpleasant taste in said product has been artificially added to said product.

The term “substance causing unpleasant taste impression” as used herein refers to substances causing unpleasant taste impression in an ingestible product, in particular, substances which taste astringent, cardboardy, dusty, dry, floury, rancid and/or metallic and/or substances that have an astringent, cardboardy, dusty, dry, floury, rancid or metallic aftertaste.

Among the unpleasant tastes or unpleasant taste impressions which can be masked according to the use of the present invention are, for example, astringency and metallic tastes.

Astringency is defined by the American Society for Testing and Materials (ASTM, 2004) as the complex of sensations due to shirking, drawing or puckering of the epithelium as a result of exposure to substances such as alums and tannins. It is believed that astringent molecules react with salivary proteins, especially proline-rich proteins, causing them to precipitate, and the resulting loss of lubricity leads to the tactile feeling associated with astringency in the mouth.

Astringent molecules are commonly plant-based products, most commonly tannins, present in fruits and leaves or bark. Some substances typically perceived as astringent are, for example, tea, red wine, rhubarb and unripe persimmons and bananas.

A typical example of substance providing astringent impression is green tea, which contains several polyphenols, known as catechins, which are known to be astringent, namely, catechin, epigallocatechin gallate, epigallocatechin, epicatechin gallate, epicatechin and their respective stereoisomers. Other examples of substances that cause astringency are proteins, such as pea protein, whey protein and soy protein. Another example of astringent taste substance are the theaflavins of black tea, namely, theaflavin, theaflavin-3-gallate, theaflavin-3′-gallate, theaflavin-3,3′-digallate, and theaflavic acid.

The taste of some substances may be perceived as a mixture of bitterness and astringency. Thus, for example, the astringent taste of green tea and black tea is sometimes perceived as a mixture of bitterness/astringency.

Other substances may have a primary taste or flavour which is not unpleasant, but have an additional unpleasant taste like bitterness, astringency or metallic notes. Without limitation, these substances may belong to the group comprising aspartame, neotame, superaspartame, saccharin, sucralose, tagatose, monellin, monk fruit, stevia extracts, individual steviol glycosides or their combinations, hernandulcin, thaumatin, miraculin, glycyrrhizin, glycyrrhetinic acid and cyclamate.

Another aspect of the invention is a process for masking the unpleasant taste of an ingestible product which contains at least one substance causing bitter, sour, pungent, or unpleasant taste impression, comprising adding to the ingestible product the compound of formula (I) according to the invention or a taste-masking composition comprising the compound of formula (I) according to the invention.

The taste-masking compound or composition according to the present invention is typically added to an ingestible product which comprises an unpleasantly tasting substance, typically, which comprises a substance causing bitter, sour, pungent, or unpleasant taste impression. The step of adding the compound or taste-masking composition to the ingestible product is meant to include adding the premixed composition, as well as incorporating the compound of formula (I) according to the invention to the ingestible product, typically followed by thoroughly mixing.

Another aspect of the invention refers to an ingestible product which comprises at least one substance causing bitter, sour, pungent, or unpleasant taste impression and a compound of formula (I) according to the invention or a composition comprising the compound of formula (I) according to the invention. Preferably, the unpleasantly tasting substance is a bitter or an astringent substance.

The amount of the unpleasantly tasting substance contained in the ingestible product should be sufficient to be perceived as unpleasant when the ingestible product does not comprise the taste-masking compound or composition according to the invention.

In one embodiment, the amount of the unpleasantly tasting substance in the ingestible product is in the range 1 ppm to 5000 ppm, based on the total weight of the ingestible product, without taste-masking compound or composition.

The compound or composition according to the invention is added to the ingestible product in a taste-masking effective amount. As used herein, a “taste-masking effective amount” is the amount that is sufficient to mask, reduce, modify or eliminate the unpleasant taste of the unpleasantly tasting substance as compared to the ingestible product without the compound or composition according to the invention. The taste masking effective amount may vary widely depending on the particular unpleasantly tasting substance, on its relative amount in the ingestible product, and on the particular ingestible product. The skilled in the art will have no difficulty in choosing the appropriate amount of the compound or composition in each particular case.

In a particular embodiment, the compound of formula (I) according to the invention or composition according to the invention is added in an amount comprised between 1 ppm to 2000 ppm, preferably comprised between 1 ppm to 1000 ppm, and more preferably comprised between 1 ppm to 500 ppm, relative to the total weight of the ingestible product. In a preferred embodiment, said amount is comprised between 10 ppm to 300 ppm, more preferably, between 50 ppm to 300 ppm, even more preferably between 100 ppm to 300 ppm relative to the total weight of the ingestible product. In an even more preferred embodiment, said amount is comprised between 100 ppm to 200 ppm relative to the total weight of the ingestible product. The total weight of the ingestible product is meant to include also the weight of the added taste-masking compound or composition.

The term “ingestible product”, as used herein, relates broadly to any substance aimed to be orally ingested, either by a human or by an animal, and includes substances which are drunk, eaten, swallowed or otherwise ingested, namely, food products and pharmaceuticals. Furthermore, the term “ingestible product” is meant to include also substances which are not intended to be ingested, but first taken into the mouth and subsequently expelled, for example, chewing gums and oral care compositions, such as mouthwashes, toothpastes, or tooth gels, for example.

In one embodiment of the present invention, the ingestible product is selected from the group consisting of food products, pharmaceuticals, and oral care compositions; preferably selected from food products and pharmaceuticals.

As it is shown in Example 1 below, the compound of formula (I) according to the invention, in particular, hesperidin, is able to reduce the bitterness of pharmaceutical products such as, for example, paracetamol solutions. Indeed, as shown in Table 2, when 100 ppm H2S is added, 20% bitterness reduction is obtained. Moreover, a bitterness reduction of 37% is obtained when 250 ppm H2S is added to the paracetamol solution.

Thus, in another embodiment of the present invention, the ingestible product is a pharmaceutical product.

In another embodiment of the present invention, the ingestible product is a food product.

The expression “food product”, as used herein, means any edible product intended for human or animal nutrition, and includes solids, semi-solids and liquids, including also beverages. The expression “food product” also includes products which are intended to be only partially ingested and subsequently expelled from the oral cavity, particularly, chewing gums.

The beverages suitable to add therein the taste-masking compound or composition according to the present invention can be, without limitation, any kind of beverage containing substance causing bitter, sour, pungent, or unpleasant taste impression. Preferably, said beverage contains a bitter substance, and it may be either carbonated or non-carbonated, either alcoholic or non-alcoholic, and include, among others, fruit-flavoured soft drinks, sodas, colas, sport drinks, and, in general, any drink containing and/or flavoured with fruits, vegetables, aromatic plants, tea, coffee or cocoa, for example; it also includes beverages containing wine or beer, for example; it also includes energy or healthy drinks containing, for example, protein hydrolysates, vitamins and/or phytonutrients having bitter and or astringent tastes. In a preferred embodiment, said beverage is a citrus-fruit juice. Preferably, said beverage is not milk or a milk-based beverage.

The beverage category includes those beverages ready to drink, as well as other forms, like powders, granulates, tablets or liquid concentrates, which are intended to be reconstituted with water.

Other food products suitable to add therein the taste-masking compound or composition according to the present invention are, for example, bakery products, such as bread, cakes, biscuits, muffins, and, in general, any kind of baked food; also dairy products such as yogurt, drinkable yogurt, frozen yogurt, cream, cheese or ice cream; soy-based products, such as soy milk or soy-lecithin; confectionary products, such as chocolate, caramels, candies, marzipan, or chewing gums; cereal-type products, such as breakfast cereals, cereal bars, energy/nutritional bars or flakes; fruit derived products, such as jam, fruit purees, preserved fruits, and sauces; vegetable derived products, such or sauces, dried vegetables, preserved vegetables or frozen vegetables; oil based products and emulsions, such as mayonnaise and several dressings, among many others, provided that they contain a substance causing bitter, sour, pungent, or unpleasant taste impression.

The food product category also includes, in particular, nutritional or dietary supplements, i.e., food products which are enriched in some nutritional ingredients, for example, vitamins, minerals, amino acids, proteins, botanicals, enzymes or other substances intended to supplement the human diet, and which may be in any suitable food form, typically as beverages or bars, for example.

Also included into the food product expression are any kinds of dried products, such as desert mixes or dried ready meals.

Food products also include, in particular, any feed intended for animal nutrition.

The pharmaceutical products suitable to add therein the taste-masking compound or composition according to the present invention are those comprising an active ingredient causing bitter, sour, pungent, or unpleasant taste impression, including as well any kind of unpleasantly tasting health supplement, for example, vitamins, minerals and mixtures thereof. Preferably, the active ingredient is selected from fluoro-quinolone antibiotics, ibuprofen, paracetamol, β-lactam antibiotics, ambroxol, guaifenesin, and mixtures thereof. Indeed, as it is shown in Example 1 below, addition of hesperidin 2S (H2S) to a paracetamol solution results in a substantial reduction of bitterness. Thus, in a particular embodiment of the invention, the active ingredient is paracetamol.

The pharmaceutical products can be in any form suitable for oral administration either for human or veterinary therapy. The pharmaceutical products can be in any form suitable for oral administration, as are well known to the skilled in the art, for example solid forms, such as tablets, chewable tablets, orally disintegrating tablets (ODT), sub-lingual tablets, orally disintegrating films (flash films), lozenges, chewable gums, or powders; or liquid forms, such as drops, syrups and suspensions; or alternatively, in the form of powders, granulates, or tablets intended to be dissolved in a liquid to be administered, for example, effervescent tablets.

The taste-masking compound or composition of the invention can be added to the ingestible product in a conventional way, as is well known to the skilled in pharmaceutical technology or food technology, for example, can be added to a pharmaceutical product, together with other excipients of the formulation, or to a food product at a suitable stage of the manufacturing process.

It is understood that the above examples are non-limiting and any ingestible product containing a substance causing bitter, sour, pungent, or unpleasant taste impression in an ingestible product, may be appropriate to add therein the compound or composition according to the present invention.

The following examples further illustrate the invention.

Further aspects and embodiments of the present invention are described in the following clauses below:
Clause 1: Use of a compound of Formula (I) or a stereoisomer or salt thereof

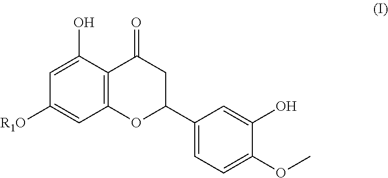

wherein R1 is hydrogen, for masking or reducing the bitter, sour, pungent, or unpleasant taste impression of a substance causing bitter, sour, pungent, or unpleasant taste impression in an ingestible product, wherein said compound of Formula (I) is not used in combination or as admixture with a hesperidin glycoside.
Clause 2: Use according to clause 1, wherein said substance is selected from the group consisting of caffeine, theobromine, theophylline, methylxanthine, quinine, brucine, strychnine, nicotine, salicin, arbutin, neohesperidin, hesperidin, naringin, quercitrin, rutin, phloridzin, pentagalloylglucose, galloylated catechols or epicatechols, proanthyocyanidines or procyanidines, thearubigin, quercetin, taxifolin, myricetin, salicin, gamma-oryzanol, caffeic acid or esters thereof, limonin, nomilin, a polymethoxylated flavone, absinthin, amarogentin, magnesium salts, calcium salts, potassium chloride, potassium gluconate, potassium carbonate, potassium sulfate, potassium lactate, potassium glutamate, potassium succinate, potassium malate, sodium sulfate, magnesium sulfate, fluoroquinolone antibiotics, paracetamol, ibuprofen, aspirin, beta-lactam antibiotics, ambroxol, propylthiouracil, guaifenesin, vitamin H, vitamin B1, vitamin B2, vitamin B6, vitamin B12, niacin, pantothenic acid, denatonium benzoate, sucralose octaacetate, leucine, isoleucine, valine, tryptophan, proline, histidine, tyrosine, lysine, phenylalanine, soy protein, whey protein, pea protein, rice protein, hemp protein, chia protein, wheat protein, potato protein, corn protein, sunflower protein, aspartame, steviol glycosides, monkfruit, collagen peptides, cannabinoids, and mixtures thereof.
Clause 3: Use according to any one of clauses 1 to 2, wherein said substance causing bitter, sour, pungent or unpleasant taste is a substance which is naturally occurring in said ingestible product.
Clause 4: Use according to clause 3, wherein said substance is a substance present in a citrus plant fruit suffering from Greening disease.
Clause 5: Use according to clause 4, wherein said substance is a polymethoxylated flavone, limonin, nomilin, hesperidin, or mixtures thereof.
Clause 6: Use according to any one of clauses 1 to 5, wherein said substance causing said bitter, sour, pungent, or unpleasant taste in said product has been artificially added to said product.
Clause 7: Use according to any one of clauses 1 to 2, wherein said ingestible product is a food product.
Clause 8: Use according to any one of clauses 1-7, wherein said food product is a beverage product.
Clause 9: Use according to clause 8, wherein said beverage is a citrus juice.
Clause 10: Use according to clause 9, wherein said citrus juice is produced from a citrus fruit plant suffering from Greening disease.
Clause 11: Use according to any one of clauses 1 to 10, wherein the compound of formula (I) or (Ia) is added in an amount of 10 ppm to 500 ppm, preferably, 100 ppm to 300 ppm, more preferably from 100 ppm to 200 ppm, by weight of the total weight of the product.
Clause 12: Use according to any one of clauses 1 to 11, wherein the reduction of said the bitter, sour, pungent, or unpleasant taste is of at least 10% when compared to the taste of the product without being added said compound of formula (I) or (Ia).

### EXAMPLES

### Taste-Masking Compositions

Hesperidin 2S (H2S) was supplied by HealthTech Bio Actives, S.L.U. (micronized; Cardiose; 93% hesperidin 2S: 7% hesperidin 2R).

Hesperidin 2S has a very low solubility in water (less than 10 ppm). However, in presence of NaOH, its solubility increases. In order to study the component in liquid matrixes, a non-heated H₂O—NaOH solution was prepared (Table 1).

TABLE 1

| | 1% sol. |
|---|---|
| Ingredients | NaOH:H₂O |
| Hesperidin 2S (micronized) | 1 g |
| NaOH | 4.2 g |
| Water | Until 100 ml |

### Example 1: Masking Bitterness of Paracetamol

The bitter masking effect of hesperidin against paracetamol was assayed. The anti-bittering effect was measured as a dose-response profile vs. a solution of paracetamol.

For evaluating the masking effect of each substance, several aqueous solutions were prepared, each containing 5000 ppm of paracetamol and different concentrations of hesperidin, prepared as indicated in Table 1, namely, 10, 50, 100, and 250.

A calibrated test panel compared each solution with several solutions of paracetamol in water at different concentrations and determined the equibitter paracetamol solution. The equibitter solution was calculated as the average of all the evaluations of the panel.

The masking effect or percentage of bitterness reduction was then calculated from the reference amount of paracetamol contained in each tested solution (5000 ppm) and the amount of paracetamol in the equibitter solution, using the following formula:

$$Masking effect = \frac{5000 - Equibitter amounrt of paracetoamol ( ppm )}{500}$$

Thus, for example, a solution containing 5000 ppm of paracetamol and 250 ppm of hesperidin was equibitter to a solution containing 4000 ppm of paracetamol. Therefore, the masking effect was 20%.

The results are shown in Table 2:

TABLE 2-1

| Hesperidin 2S | Equibitter paracetamol | % of bitterness |
|---|---|---|
| (ppm) | concentration (ppm) | reduction |
| 10 | 4750 | 5 |
| 50 | 4450 | 11 |
| 100 | 4000 | 20 |
| 250 | 3150 | 37 |

The results in Table 2-1 show that hesperidin is able to reduce the bitterness of paracetamol solutions. When 100 ppm H2S is added, 20% bitterness reduction is obtained. A bitterness reduction of 37% is obtained when 250 ppm H2S is added.

The bitter masking effect of a racemic hesperidin (57% hesperidin 2S: 43% hesperidin 2R; purchased by HealthTech Bio Actives, S.L.U. Beniel, Spain) against paracetamol was also assayed. The anti-bittering effect was measured as a dose-response profile vs. a solution of paracetamol as described above.

TABLE 2-2

| Racemic | | |
|---|---|---|
| Hesperidin | Equibitter paracetamol | % of bitterness |
| (ppm) | concentration (ppm) | reduction |
| 10 | 4800 | 4 |
| 50 | 4700 | 6 |
| 100 | 4350 | 13 |
| 250 | 4100 | 18 |

The results show that non-racemic hesperidin (93% hesperidin 2S: 7% hesperidin 2R) was able to reduce paracetamol bitterness to a greater extent when compared with the bitterness reduction obtained with the racemic hesperidin. Indeed, when 100 ppm hesperidin was tested, percentage of bitterness reduction was 13% with the racemic versus 20% with the non-racemic. Moreover, when 250 ppm H2S was tested, a reduction of 18% was obtained with racemic hesperidin versus a 37% reduction when non-racemic hesperidin 2S was used.

Further, the bitter masking effect of a different flavonoid compound, neodiosmin (purchased by HealthTech Bio Actives, S.L.U. Beniel, Spain), was also tested. The anti-bittering effect was measured as a dose-response profile vs. a solution of paracetamol as explained before.

TABLE 2-3

| | Equibitter paracetamol | % bitterness |
|---|---|---|
| Neodiosmin | concentration | reduction |
| (ppm) | (ppm) | (%) |
| 50 | 4400 | 12 |
| 100 | 4400 | 12 |
| 250 | 4200 | 16 |
| 500 | 3800 | 24 |

The results in Table 2-3 show that neodiosmin is able to reduce the bitterness of paracetamol solutions to a lesser extent than hesperidin 2S. When 100 ppm neodiosmin is added, 12% bitterness reduction is obtained whereas a 20% bitterness reduction is obtained when 100 ppm H2S is added. Also, 250 ppm neodiosmin produces a 16% bitterness reduction versus 37% when 250 ppm H2S is added.

### Example 2: Evaluation of Hesperidin 2S in Orange Juices Containing Oleuropeins

NaOH:H₂O solutions containing different concentrations (100 ppm and 200 ppm) of Hesperidin 2S were prepared as explained above (Table 1) and added to orange juices containing 500 ppm Olews 40% (500 ppm Olews 40%=160 ppm oleuropein). Olews 40% provided by HealthTech Bio Actives S.L.U. (Beniel, Spain).

TABLE 3

| Ingredients | Control | Trial 1 | Trial 2 |
|---|---|---|---|
| Hesperidin 2S | — | 2.5 g | 5 g |
| (1% sol. NaOH:H₂O) | | (100 ppm) | (200 ppm) |
| Olews 40% | — | 0.5 g | 0.5 g |
| (sol. 25% H₂O) | | (500 ppm) | (500 ppm) |
| Orange Juice | Until 250 mL | Until 250 mL | Until 250 mL |

The taste masking effect against oleuropeins was assayed following an analogous procedure to the one used in Example 1 for paracetamol.

For each tested solution, a test panel determined the equibitter oleuropein solution, and the masking effect was calculated as explained in Example 1. A panel of experts was used to determine the reduction in unpleasant taste impressions of the orange juice preparations containing oleuropeins after addition of H2S.

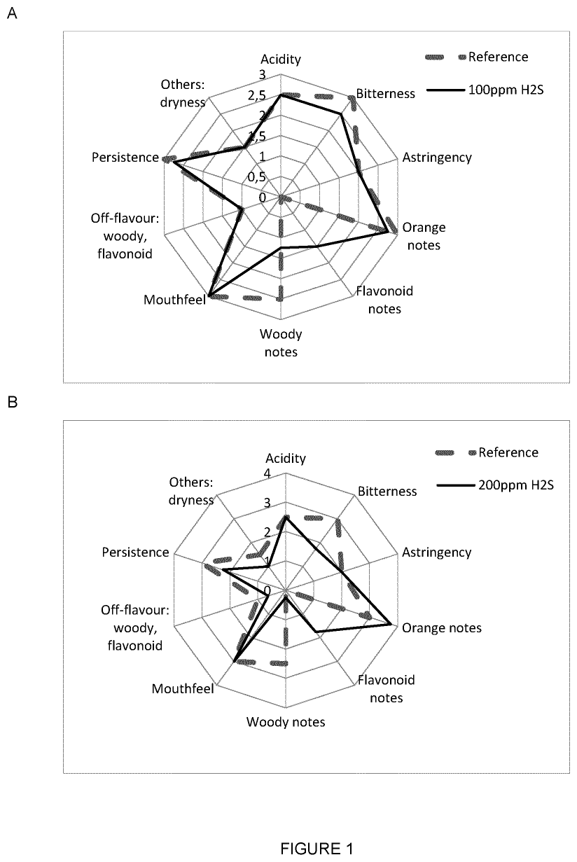

The results showed that when 200 ppm H2S were added, the woody and bitter taste in the orange juice preparations containing oleuropein were masked (FIG. 1A).

Moreover, when 200 ppm H2S were added, the woody and bitter taste coming from the oleuropein were significantly masked (40%) and also the orange notes were enhanced (FIG. 1B).

### Example 3: Evaluation of Hesperidin 2S in Crackers With Reduced NaCl

A dosage of 200 ppm of H2S was added to crackers with reduced content of sodium chloride (NaCl) and containing potassium chloride (KCl; Panreac). The composition of the crackers is disclosed in Table 4 below:

TABLE 4

| Ingredients | Control | | H2S (200 ppm) | |
|---|---|---|---|---|
| Flour | 184 | g | 184 | g |
| Cold Water | 95 | g | 95 | g |
| Sunflower oil | 65 | g | 65 | g |
| Sugar | 7 | g | 7 | g |
| KCl | 6.3 | g | 6.3 | g |
| NaCl | 5.25 | g | 5.25 | g |
| Baking powder | 5 | g | 5 | g |
| Large egg | 1 | | 1 | |
| H2S (dry powder) | — | | 0.1 | g |

The taste masking effect was assayed, following an analogous procedure to the one disclosed in Example 1. A panel of experts was used to determine the reduction in unpleasant taste impressions of the potassium chloride-containing preparations after addition of H2S.

The results showed a significant improvement compared to the reference. As it is shown in FIG. 2, the pungent and metallic notes of the KCl-containing crackers, were suppressed. Moreover, the addition of H2S to the crackers with reduced content of sodium chloride, resulted in an increase of saltiness and overall giving a more rounded taste of the cracker.

### Example 4: Evaluation of Hesperidin 2S in Dark Chocolate 95%

Reduction of unpleasant taste impressions in dark chocolate (95%) preparations was evaluated using different concentrations of H2S (100 ppm and 200 ppm).

Hesperidin 2S (dry powder) was incorporated into a sample of melted dark chocolate (95%). The liquid mass was cooled and mold, obtaining a solid chocolate.

TABLE 5

| Ingredients | Control | Trial 1 | Trial 2 |
|---|---|---|---|
| H2S | — | 0.005 g | 0.01 g |
| | | (100 ppm) | (200 ppm) |
| Dark | 50 g | 50 g | 50 g |
| chocolate | | | |
| 95% | | | |

A panel of experts was used to determine the reduction in unpleasant taste impressions of the dark chocolate (95%) preparations.

As it can be seen in FIG. 3, addition of 200 ppm of H2S resulted in a decrease in bitterness, whereas cocoa notes were slightly enhanced.

Moreover, addition of 200 ppm of H2S produced a significant improvement. In this case, bitterness and astringency were decreased and flavonoid notes were not present (see FIG. 3).

### Example 5: Evaluation of Hesperidin 2S in Limonin Solution

Citrus greening disease (Huanglongbing or HLB) is a disease of citrus caused by a vector-transmitted pathogen. The causative agents are motile bacteria, Candidatus Liberibacter spp.

Affected trees have stunted growth, bear multiple off-season flowers (most of which fall off), and produce small, irregularly shaped fruit with a thick, pale peel that remains green at the bottom and tastes very bitter. One the main causes of the characteristic bitter taste, is the increase of two molecules: limonin and nomilin.

In order to determine the reduction in unpleasant taste impressions (bitterness) by hesperidin in a greening disease model, a limonin solution was prepared as indicated in Table 6. This solution was used for testing. Limonin (99.66%) was purchased from TargetMol.

TABLE 6

| Ingredients | Sol. 1% Limonin |
|---|---|
| Limonin | 0.1 g |
| Ethanol 96% | 50 g |

The effect of H2S (200 ppm) in a limonin solution (50 ppm) was evaluated. A panel of experts was used to determine the reduction in unpleasant taste impressions of the limonin-containing preparation.

The results show (Table 7) that a dosage of 200 ppm of H2S produced a substantial debittering effect (30% bitterness reduction).

TABLE 7

| | Equibitter | % of bitterness reduction |
|---|---|---|
| Limonin 50 ppm + 200 ppm | 35 ppm | 30 |
| H2S | | |

### Example 6: Evaluation of Hesperidin 2S in Greening Affected Orange Juices

Orange Juice samples obtained from oranges from trees afflicted with greening disease were provided by F&M Solutions (Brasil). The juice samples were prepared by squeezing oranges collected from afflicted tree branches from local Brazilian farmers. The squeezed orange juice was bottled and pasteurized by standard methods well known in the art.

Different concentrations of Hesperidin 2S (100 ppm and 200 ppm) were evaluated in the orange juices from greening afflicted oranges.

TABLE 8

| Ingredients | Control | Trial 1 | Trial 2 |
|---|---|---|---|
| H2S (1% sol. | — | 1 g | 2 g |
| NaOH:H2O) | | (100 ppm) | (200 ppm) |
| Pasteurized | 100 ml | Until 100 ml | Until 100 ml |
| Orange Juice | | | |

A panel of experts was used to determine the reduction in unpleasant taste impressions of the different samples.

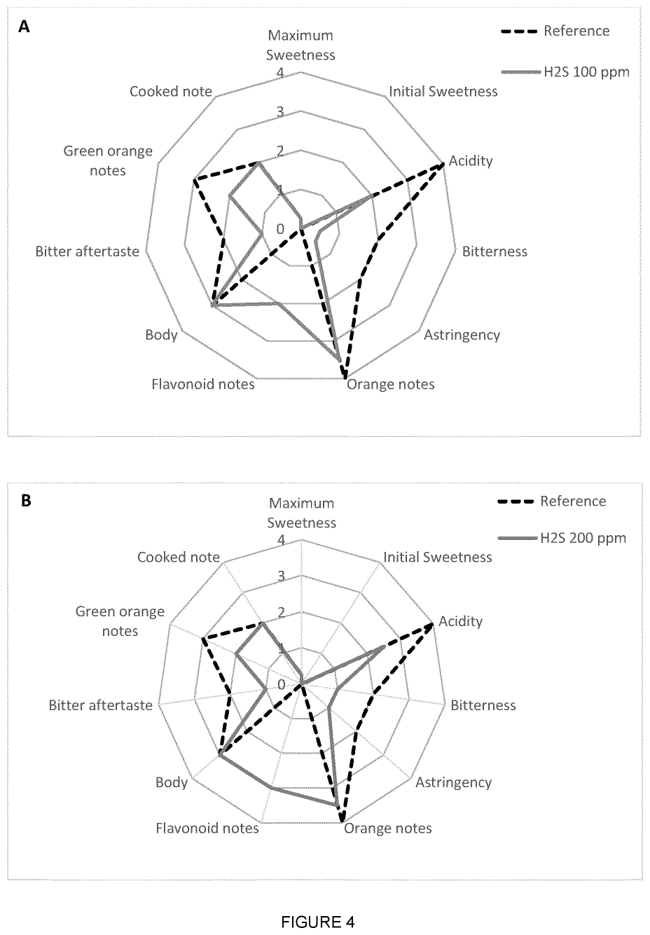

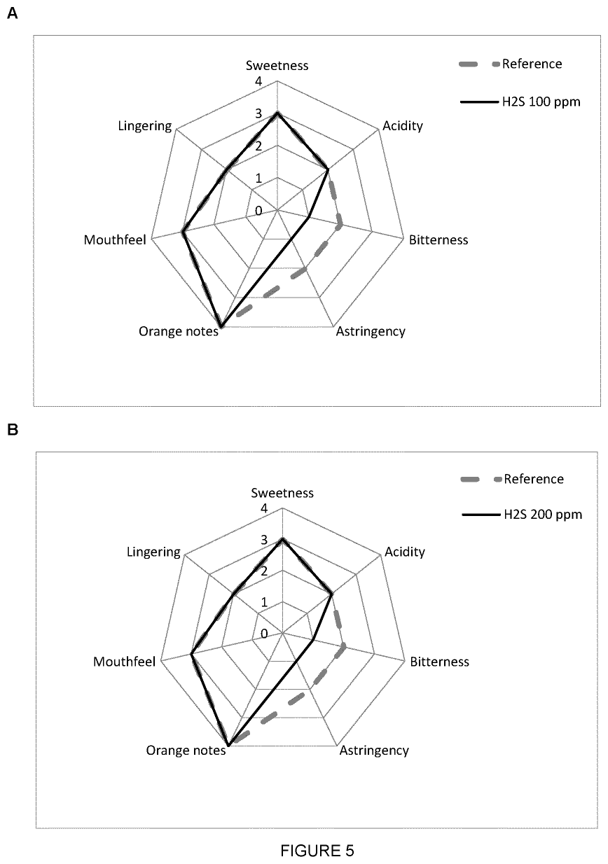

The results show (FIG. 4A and FIG. 4B) that the control sample (greening affected orange juice) have no significant sweetness but high acidity, astringency and bitterness, leaving a lingering bitter aftertaste. High orange notes, with noticeable green orange notes were detected.

Addition of 100 ppm of H2S produced a significant reduction of bitterness, acidity and astringency. Reduction in green orange notes and bitter aftertaste was also detected. Further, there is a slight reduction in overall orange notes but there is a noticeable flavonoid note.

Addition of 200 ppm of H2S also produced a reduction of bitterness, acidity and astringency, though to a lesser extent. There is also a reduction of green orange notes and bitter aftertaste. There is a reduction in overall orange notes and increased flavonoid note.

### Example 7: Evaluation of Hesperidin 2S in Fresh Squeezed Orange Juices

NaOH:H₂O solutions containing different concentrations (100 ppm and 200 ppm) of Hesperidin 2S were prepared as explained above (Table 1) and added to fresh squeezed orange juices.

TABLE 9

| Ingredients | Control | Trial 1 | Trial2 |
|---|---|---|---|
| Hesperidin 2S (1% sol. | — | 2.5 g | 5 g |
| NaOH:H₂O) | | (100 ppm) | (200 ppm) |
| Fresh squeezed | Until 250 mL | Until 250 mL | Until 250 mL |
| Orange Juice | | | |

A panel of experts was used to determine the reduction in unpleasant tastes of the fresh squeezed orange juices after addition of different concentrations of H2S.

The results showed that when 100 ppm or 200 ppm H2S were added, the astringent and bitter taste in the orange juices were clearly reduced (FIGS. 5A and 5B).

### Example 8: Evaluation of hesperidin 2S in tonic water

NaOH:H₂O solutions containing different concentrations (100 ppm and 200 ppm) of Hesperidin 2S were prepared as explained above (Table 1) and added to a tonic water samples prepared as indicated in Table 10 below:

TABLE 10

| Ingredients | Control | Trial 1 | Trial 2 |
|---|---|---|---|
| Sucrose | 8.2 g | 8.2 g | 8.2 g |
| Citric acid | 0.6 g | 0.6 g | 0.6 g |
| Lemon-lime | 0.1 g | 0.1 g | 0.1 g |
| flavour | | | |
| Quinine | 0.0078 g | 0.0078 g | 0.0078 g |
| Hydrocloride | (78 ppm) | (78 ppm) | (78 ppm) |
| Dihydrate | | | |
| H2S (1% sol | — | 1 g | 2 g |
| NaOH:H2O) | | (100 ppm) | (200 ppm) |
| Water | Until 100 mL | Until 100 mL | Until 100 mL |

A panel of experts was used to determine the reduction in unpleasant taste impression of the different samples.

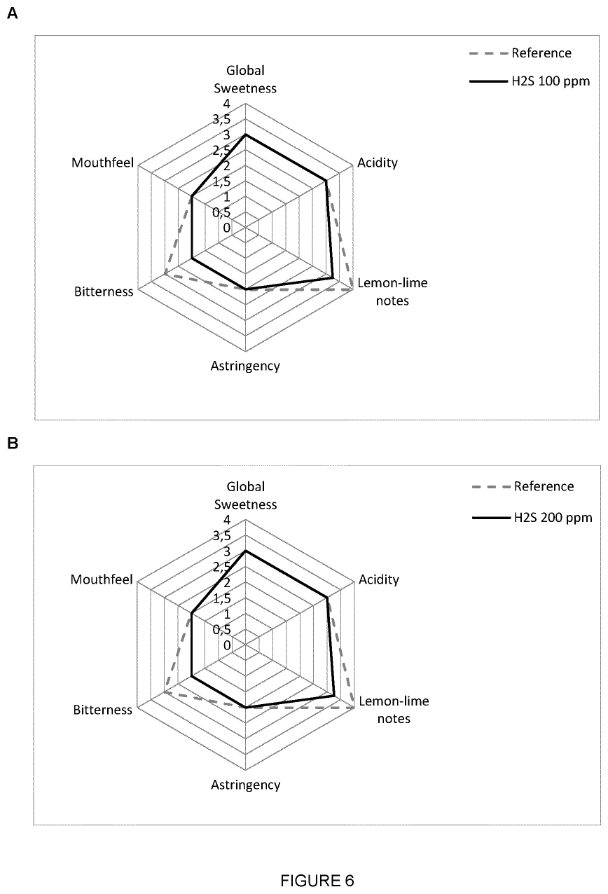

The results show (FIG. 6A and FIG. 6B) that addition of 100 ppm of H2S produced a significant reduction of bitterness. Addition of 200 ppm of H2S also produced a reduction of bitterness.

### Example 9: Evaluation of Hesperidin 2S in Plant-Based Burger Containing Oleuropeins

Reduction of unpleasant taste impression in a plant-based burger was evaluated using different concentrations of H2S (100 ppm and 200 ppm). Hesperidin 2S (dry powder) was incorporated into the preparations containing 2000 ppm Olews 40% provided by HealthTech Bio Actives, S.L.U. (Beniel, Spain) (2000 ppm Olews 40%=600 ppm oleuropein) as indicated in Table 11 below:

TABLE 11

| | Control | Trial 1 | Trial 2 |
|---|---|---|---|
| Ingredients | % (w/w) | | |
| Water | 63 g | 63 g | 63 g |
| Soy Protein texturates | 15 g | 15 g | 15 g |
| Sunflower oil | 10 g | 10 g | 10 g |
| Pea Protein | 8 g | 8 g | 8 g |
| Methyl cellulose | 1.9 g | 1.9 g | 1.9 g |
| Salt | 0.7 g | 0.7 g | 0.7 g |
| Beef flavour | 0.5 g | 0.5 g | 0.5 g |
| Caramel colour | 0.4 g | 0.4 g | 0.4 g |
| Olews 40% | 0.2 g | 0.2 g | 0.2 g |
| Hesperidin 2S | — | 0.01 g | 0.02 g |
| | | (100 ppm) | (200 ppm) |

A panel of experts was used to determine the reduction in unpleasant taste impressions of the plant-based burger preparations.

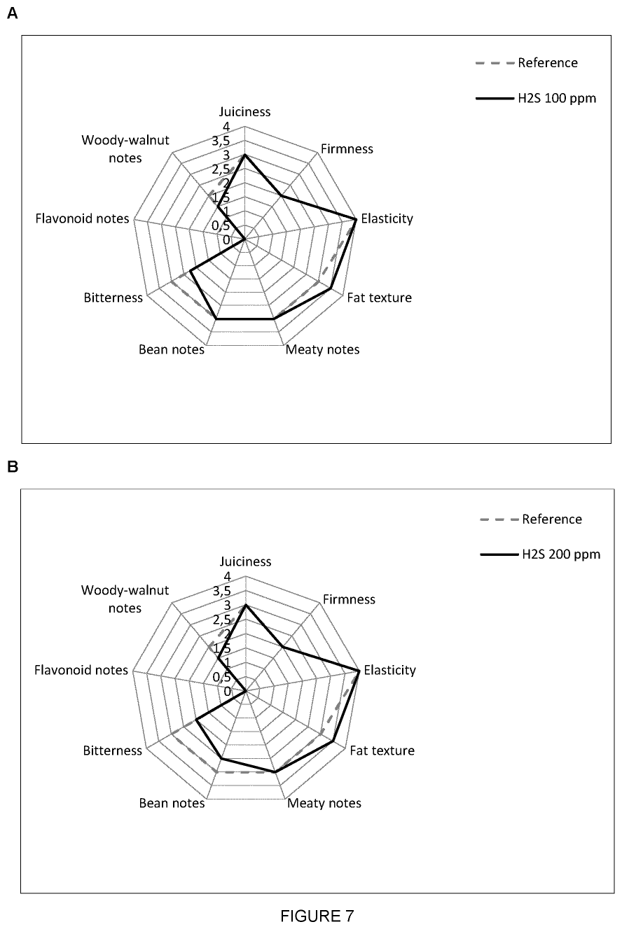

As it can be seen in FIG. 7A, addition of 100 ppm of H2S resulted in a decrease in bitterness. Also, woody off-flavor notes are reduced. Further, flavonoid notes were not present (see FIG. 7A).

Addition of 200 ppm of H2S produced a significant improvement. In this case, bitterness and bean notes were decreased. Woody off-flavor notes were also reduced. Further, flavonoid notes were not present (see FIG. 7B).

### Example 10: Evaluation of Hesperidin 2S in Protein Milkshake

NaOH:H₂O solutions containing different concentrations (100 ppm and 200 ppm) of Hesperidin 2S were prepared as explained above (Table 1) and added to a protein milkshake prepared as indicated in Table 12 below.

TABLE 12

| Ingredients | Control | Trial 1 | Trial 2 |
|---|---|---|---|
| Sucrose | 7.5 g | 7.5 g | 7.5 g |
| Cocoa | 1 g | 1 g | 1 g |
| Pea Protein | 7.5 g | 7.5 g | 7.5 g |
| H2S (1% sol | — | 1 g | 2 g |
| NaOH:H2O) | | (100 ppm) | (200 ppm) |
| Skimmed milk | Until 100 mL | Until 100 mL | Until 100 mL |

The taste masking effect was assayed, following an analogous procedure to the one used in Example 1. A panel of experts was used to determine the reduction in unpleasant taste impressions of the protein milkshake preparations after addition of H2S.

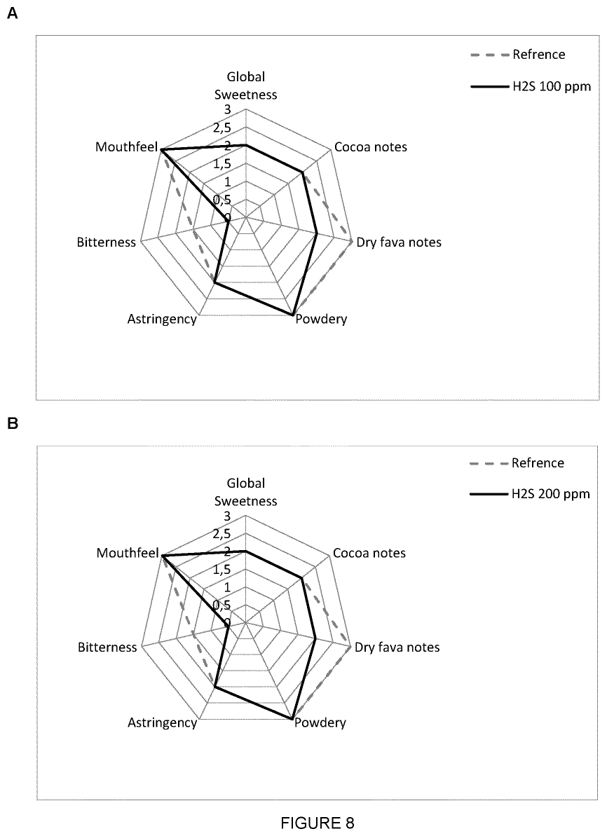

The results showed that when 100 ppm or 200 ppm H2S were added, bitter taste is reduced. Further, the dry fava notes were also reduced (FIG. 8).

1-17. (canceled)

18. A method for masking or reducing the bitter, sour, pungent, or unpleasant taste impression of a substance causing bitter, sour, pungent, or unpleasant taste impression in an ingestible product, the method comprising incorporating a compound of Formula (I) or a stereoisomer or salt thereof into said ingestible product, 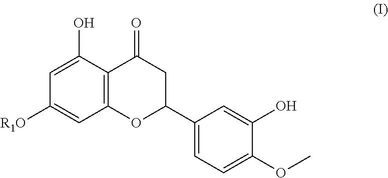
wherein R1 is a saccharide consisting of 1 or 2 monosaccharide units;
wherein said compound of Formula (I) is not used in combination or as admixture with a hesperidin glycoside.

19. The method according to claim 18, wherein said compound of Formula (I) is (Ia) 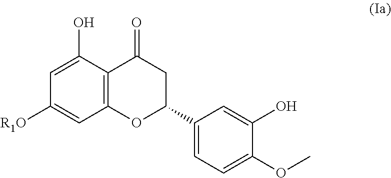
wherein R1 is a saccharide consisting of 2 monosaccharide units.

20. The method according to claim 19, wherein said compound of formula (Ia) is (2S)-5-hydroxy-2-(3-hydroxy-4-methoxyphenyl)-7-[(2S,3R,4S,5S,6R)-3,4,5-trihydroxy-6-{[(2R,3R,4R,5R,6S)-3,4,5-trihydroxy-6-methyloxan-2-yl]oxymethyl}oxan-2-yl]oxy-2,3-dihydrochromen-4-one.

21. The method according to claim 19, wherein said compound is used as a taste masking composition in which the (2S)- and (2R)-enantiomers are present in a weight ratio of 99:1 to 80:20.

22. The method according to claim 18, wherein said substance is caffeine, theobromine, theophylline, methylxanthine, quinine, brucine, strychnine, nicotine, salicin, arbutin, neohesperidin, hesperidin, naringin, quercitrin, rutin, phloridzin, pentagalloylglucose, galloylated catechols or epicatechols, proanthyocyanidines or procyanidines, thearubigin, quercetin, taxifolin, myricetin, salicin, gamma-oryzanol, caffeic acid or esters thereof, limonin, nomilin, a polymethoxylated flavone, absinthin, amarogentin, magnesium salts, calcium salts, potassium chloride, potassium gluconate, potassium carbonate, potassium sulfate, potassium lactate, potassium glutamate, potassium succinate, potassium malate, sodium sulfate, magnesium sulfate, fluoroquinolone antibiotics, paracetamol, ibuprofen, aspirin, beta-lactam antibiotics, ambroxol, propylthiouracil, guaifenesin, vitamin H, vitamin B1, vitamin B2, vitamin B6, vitamin B12, niacin, pantothenic acid, denatonium benzoate, sucralose octaacetate, leucine, isoleucine, valine, tryptophan, proline, histidine, tyrosine, lysine, phenylalanine, soy protein, whey protein, pea protein, rice protein, hemp protein, chia protein, wheat protein, potato protein, corn protein, sunflower protein, aspartame, steviol glycosides, monkfruit, collagen peptides, cannabinoids, or mixtures thereof.

23. The method according to claim 22, wherein said substance causing bitter, sour, pungent or unpleasant taste is a substance which is naturally occurring in said ingestible product.

24. The method according to claim 23, wherein said substance is a substance present in a citrus plant fruit suffering from Greening disease.

25. The method according to claim 24, wherein said substance is a polymethoxylated flavone, limonin, nomilin, hesperidin, or mixtures thereof.

26. The method according to claim 18, wherein said substance causing said bitter, sour, pungent, or unpleasant taste in said product has been artificially added to said product.

27. The method according to claim 18, wherein said ingestible product is a food product.

28. The method according to claim 27, wherein said food product is a beverage product.

29. The method according to claim 28, wherein said beverage is a citrus juice.

30. The method according to claim 29, wherein said citrus juice is produced from a citrus fruit plant suffering from Greening disease.

31. The method according to claim 18, wherein the compound of formula (I) is added in an amount of 10 ppm to 500 ppm, by weight of the total weight of the product.

32. The method according to claim 18, wherein the reduction of the bitter, sour, pungent, or unpleasant taste is of at least 10% when compared to the taste of the product without being added said compound of formula (I).

33. Taste masking composition comprising a compound of formula (I) 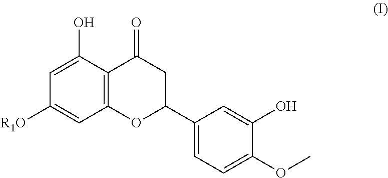
wherein R1 is saccharide consisting of 2 monosaccharide units and the (2S)- and (2R)-enantiomers are present in a weight ratio of 99:1 to 80:20, and wherein said composition does not contain a hesperidin glycoside.

34. The taste masking composition according to claim 33, wherein said compound of formula (I) is hesperidin.

35. The method according to claim 20, wherein said compound is a taste masking composition in which the (2S)- and (2R)-enantiomers are present in a weight ratio of 99:1 to 80:20.

36. The method according to claim 20, wherein said substance is caffeine, theobromine, theophylline, methylxanthine, quinine, brucine, strychnine, nicotine, salicin, arbutin, neohesperidin, naringin, quercitrin, rutin, phloridzin, pentagalloylglucose, galloylated catechols or epicatechols, proanthyocyanidines or procyanidines, thearubigin, quercetin, taxifolin, myricetin, salicin, gamma-oryzanol, caffeic acid or esters thereof, limonin, nomilin, a polymethoxylated flavone, absinthin, amarogentin, magnesium salts, calcium salts, potassium chloride, potassium gluconate, potassium carbonate, potassium sulfate, potassium lactate, potassium glutamate, potassium succinate, potassium malate, sodium sulfate, magnesium sulfate, fluoroquinolone antibiotics, paracetamol, ibuprofen, aspirin, beta-lactam antibiotics, ambroxol, propylthiouracil, guaifenesin, vitamin H, vitamin B1, vitamin B2, vitamin B6, vitamin B12, niacin, pantothenic acid, denatonium benzoate, sucralose octaacetate, leucine, isoleucine, valine, tryptophan, proline, histidine, tyrosine, lysine, phenylalanine, soy protein, whey protein, pea protein, rice protein, hemp protein, chia protein, wheat protein, potato protein, corn protein, sunflower protein, aspartame, steviol glycosides, monkfruit, collagen peptides, cannabinoids, or mixtures thereof.

37. A method for masking or reducing the bitter, sour, pungent, or unpleasant taste impression of a substance causing bitter, sour, pungent, or unpleasant taste impression in an ingestible product, the method comprising incorporating a compound of Formula (I) or a stereoisomer or salt thereof into said ingestible product, 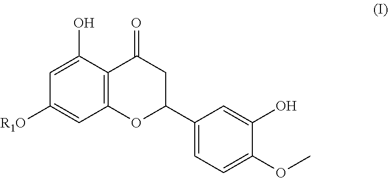
wherein R1 is a saccharide consisting of 1 or 2 monosaccharide units;
wherein said compound of Formula (I) is not used in combination or as admixture with a hesperidin glycoside, and
providing said ingestible product to a subject for ingestion.
